(19) United States
(12) Patent Application Publication
(10) Pub. No.: US 2007/0225729 A1
(43) Pub. Date: Sep. 27, 2007

(54) Medical device basket legs of a plurality of materials

(76) Inventor: Eric Cheng, Bloomington, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/384,518

(22) Filed: Mar. 21, 2006

(51) Int. Cl. A61B 17/22

(52) U.S. Cl. 606/127

(57) ABSTRACT

A medical device includes a sheath defining a lumen, an elongate member disposed within the lumen, and a basket connected to a distal end of the elongate member. The basket is retractable within and extendable from the lumen. The basket includes a plurality of legs and each of the plurality of legs includes a first elongate structure of a first material braided with a second elongate structure of a second material. The second material has a resistance to laser energy greater than a resistance to laser energy of the first material.

### FIELD OF THE INVENTION

This disclosure relates generally to medical devices and, more particularly, to devices for the capture and/or retrieval of objects within a body, such as stones, other calculi, or foreign matter.

### BACKGROUND OF THE INVENTION

Extractors have been used to remove stones and other calculi from within the body of a patient. One type of extractor includes a sheath, an elongate member extending within and moveable relative to the sheath, and a basket connected to the distal end of the elongate member. The basket may have a number of legs, and may have a collapsed position within the sheath and an expanded position beyond a distal end of the sheath. When the basket is in the expanded position, a targeted stone may be captured within the basket and removed from the body. Some stones, however, may be too large to be removed from the body after being captured within the basket. In this situation, a device, such as a laser fiber or other laser lithotripsy device, may be used to break up the stone while the stone is captured. The stone and/or stone particles may then be removed from the body without harming the surrounding body tissue.

It may be difficult, however, to break up the stone with such a lithotripsy device without accidentally cutting or otherwise damaging the legs of the basket. Such damage may cause the user to lose retention of the stone. Such damage may also make the basket difficult to extract from the body without causing trauma to the surrounding body tissue.

The present disclosure provides retrieval devices and methods of using the same that avoid some or all of the aforementioned shortcomings of existing devices.

### SUMMARY OF THE INVENTION

In accordance with an exemplary embodiment of the present disclosure, a medical device includes a sheath defining a lumen, an elongate member disposed within the lumen, and a basket connected to a distal end of the elongate member. The basket is retractable within and extendable from the lumen. The basket includes a plurality of legs, and each of the plurality of legs includes a first elongate structure of a first material braided with a second elongate structure of a second material. The second material has a resistance to laser energy greater than a resistance to laser energy of the first material.

In accordance with certain embodiments of the present disclosure, the device may include one or more of the following features. For example, the first material comprises a metal, and the metal is one of nitinol, stainless steel, cobalt chromium, nickel, and titanium. The first elongate structure is a wire. The second material is not a metal. Instead, the second material is one of a ceramic material and a polymer, and the second elongate structure is a fiber. The basket forms a predetermined shape in an expanded position, and the predetermined shape is one of substantially spherical and substantially lemon-shaped. The medical device further includes an atraumatic tip formed at a distal end of the basket. The elongate member comprises the first elongate structure and the second elongate structure of at least one of the plurality of legs. In an exemplary embodiment, the basket comprises at least three legs, and at least one of the plurality of legs comprises at least two ceramic fibers and at least one metallic wire. In another exemplary embodiment, at least one of the plurality of legs comprises at least two ceramic fibers and at least two metallic wires.

In accordance with another exemplary embodiment of the present disclosure, a method of removing matter from the body of a patient includes providing a medical device including a sheath defining a lumen, an elongate member disposed within the lumen, and a basket connected to a distal end of the elongate member. The basket is retractable within and extendable from the lumen. The basket includes a plurality of legs, and each of the plurality of legs includes a first elongate structure of a first material braided with a second elongate structure of a second material. The second material has a resistance to laser energy greater than a resistance to laser energy of the first material. The method further includes advancing the medical device to a treatment site within the body of the patient and capturing the matter within the basket of the device. The method also includes reducing the size of the matter and removing the medical device from the body of the patient.

In accordance with certain embodiments of the present disclosure, the method may include one or more of the following features. For example, the method includes immobilizing the matter with the plurality of legs of the basket and advancing a laser fiber to the treatment site to assist in reducing the size of the matter. The method also includes preventing particles of the matter from escaping from the treatment site with the plurality of legs of the basket and sweeping at least a portion of the matter from the treatment site with the plurality of legs of the basket. In the method described above, the matter is one of a kidney stone, a struvite, a uric acid stone, a cystine stone, and a solid deposit. In addition, the first material comprises a metal. The metal is one of nitinol, stainless steel, cobalt chromium, nickel, and titanium, and the first elongate structure is a wire. Moreover, the second material is not a metal, and the second material comprises one of a ceramic material and a polymer. The second elongate structure is a fiber.

In accordance with a further exemplary embodiment of the present disclosure, a medical retrieval device basket includes a plurality of interconnected legs. Each of the legs includes a first material braided with a second material. The second material has a resistance to laser energy greater than a resistance to laser energy of the first material. The first material includes a metal, and the metal is one of nitinol, stainless steel, cobalt chromium, nickel, and titanium. The second material is not a metal. Instead, the second material includes one of a ceramic material and a polymer. The basket forms a predetermined shape in an expanded position, and the predetermined shape is one of substantially spherical and substantially lemon-shaped. The medical retrieval device basket further includes an atraumatic tip formed at a distal end of the basket. The basket includes at least three legs and each of the plurality of legs includes at least two strands of the second material braided with at least one strand of the first material. In another embodiment, each of the plurality of legs includes at least two strands of the second material braided with at least two strands of the first material.

Moreover, in an embodiment, the first material of each of the plurality of legs comprises a strip formed from a sheath. The strip is formed by removing at least a portion of the sheath. The second material includes one of a ceramic material and a polymer. The first material is a metal wire and the second material is a non-metal strand.

It is to be understood that both the foregoing general description and the following detailed description are exemplary and explanatory only, and are not restrictive of the invention as claimed.

### BRIEF DESCRIPTION OF THE DRAWINGS

FIG. 1 is a partial cross-sectional view of a retrieval device according to an exemplary embodiment of the present disclosure.

FIG. 2 illustrates an alternate configuration of the device of FIG. 1.

FIG. 3 is a cross-sectional view of a basket leg according to an exemplary embodiment of the present disclosure.

FIG. 4 is a cross-sectional view of a basket leg according to another exemplary embodiment of the present disclosure.

FIG. 5 is a cross-sectional view of a basket leg according to still another exemplary embodiment of the present disclosure.

FIG. 6 is a side view of the basket leg of FIG. 3.

FIG. 7 is a side view of a retrieval device according to another exemplary embodiment of the present disclosure.

FIG. 8 illustrates a basket leg of the retrieval device of FIG. 7.

FIG. 9 is a partial view of the retrieval device of FIG. 1.

### DETAILED DESCRIPTION

Exemplary embodiments of the present disclosure are illustrated in the accompanying drawings. Wherever possible, the same reference numbers will be used throughout the drawings to refer to the same or like parts.

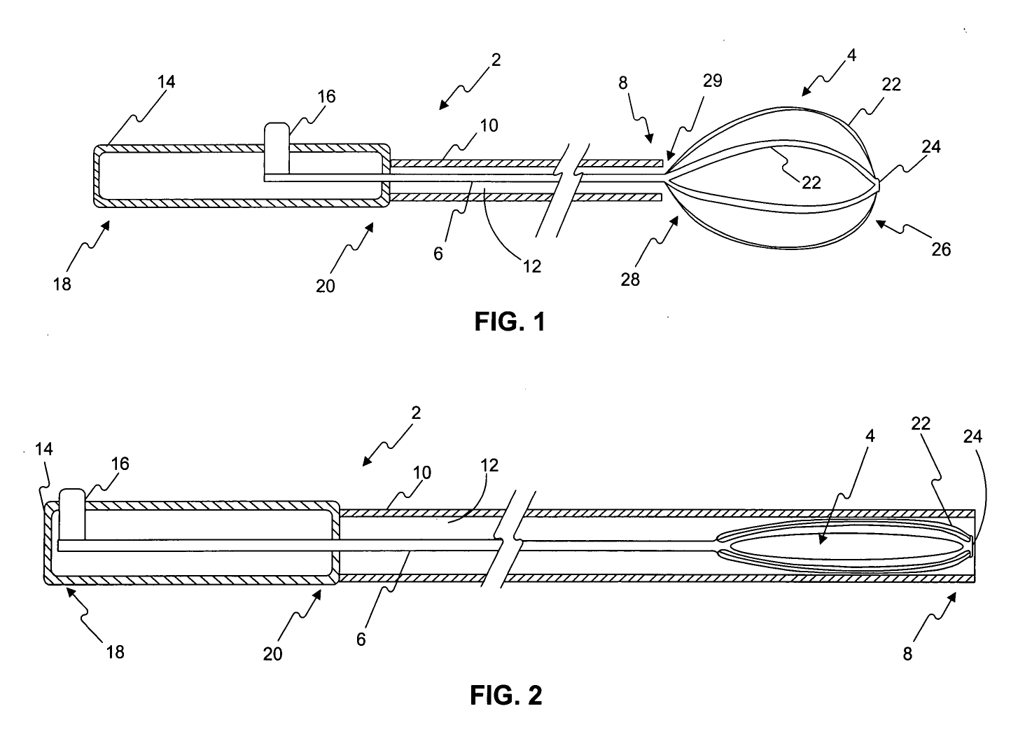

FIG. 1 illustrates a stone retrieval device 2 according to an exemplary embodiment of the present disclosure. The device 2 includes a basket 4 connected to an elongate member 6. The device 2 further includes a sheath 10 defining a lumen 12, and the elongate member 6 is disposed within the lumen 12. Relative movement between the elongate member 6 and the sheath 10 assists the basket 4 in forming an expanded position (shown in FIG. 1) where the basket 4 is disposed beyond a distal end 8 of the sheath 10, and a retracted position (shown in FIG. 2) where the basket 4 is disposed within the lumen 12 of the sheath 10.

The basket 4 may include a plurality of legs 22. The legs 22 and, thus, a proximal end 28 of the basket 4 may be connected to a distal end 29 of the elongate member 6 in any conventional way. As will be described below, in an exemplary embodiment, the legs 22 may be formed of the same piece or pieces of material as the elongate member 6. Although FIGS. 1 and 2 show a basket 4 having four legs 22, other exemplary embodiments of the basket 4 may include more or fewer than four legs 22 to facilitate the retrieval of a stone, calculi, or other foreign matter. The basket 4 may further include an atraumatic tip 24 at a distal end 26 of the basket 4.

The device 2 may further include a handle 14 configured to assist in transitioning the basket 4 between the expanded position and the retracted position. The handle 14 may include, for example, a thumb slide 16 or other conventional mechanisms configured to assist in manipulating the position of the basket 4. In an exemplary embodiment, moving the thumb slide 16 toward a distal end 20 of the handle 14 may move the elongate member 6 and the basket 4 in a distal direction relative to the handle 14 and, thus, assist in transitioning the basket 4 to the expanded position shown in FIG. 1. In such an embodiment, moving the thumb slide 16 toward a proximal end 18 of the handle 14 may move the elongate member 6 and the basket 4 in a proximal direction relative to the handle 14 and, thus, assist in transitioning the basket 4 to the retracted position shown in FIG. 2. In this exemplary embodiment, the sheath 10 may remain stationary with respect to the handle 14. In an additional exemplary embodiment, the elongate member 6 and the basket 4 may remain stationary with respect to the handle 14, and the thumb slide 16 may be configured to move the sheath 10 relative to the basket 4.

The elongate member 6 may be formed from, for example, a wire, rod, tube, hypotube, cannula, stent, or other piece of biocompatible material or combination of biocompatible materials known in the art. Such materials may include, but are not limited to, polyamide, PEBAX, stainless steel (such as 300 and 400 series), cobalt chromium, nickel, titanium, nitinol, thermoforming plastic, polytetrafluoroethylene (“PTFE”), and expanded polytetrafluoroethylene (“ePTFE”). The elongate member 6 may also be a metal coated with a polymer and may have one or more layers of material. The elongate member 6 may be solid or hollow, and may be substantially cylindrical. Alternatively, the elongate member 6 may be formed from a flat sheet of material. If formed from a flat sheet, the elongate member 6 may be formed into a substantially cylindrical shape.

The overall length and diameter of the elongate member 6 may vary depending on the application. For example, a relatively long elongate member 6 may be advantageous for retrieving stones or other calculi deep within the body of the patient. In addition, an elongate member 6 having a relatively small diameter may be advantageous for retrieving stones from restricted passageways within the human urinary tract. The elongate member 6 may be relatively flexible to facilitate the retrieval of stones located in complex body structures.

The sheath 10 may be formed from any of the materials discussed above with respect to the elongate member 6. Although FIGS. 1 and 2 illustrate a sheath 10 having a single lumen 12, in additional exemplary embodiments of the present disclosure, the sheath 10 may define more than one lumen 12. The sheath 10 may be dimensioned to fully enclose the elongate member 6 and the basket 4 when the basket 4 is in the retracted position shown in FIG. 2.

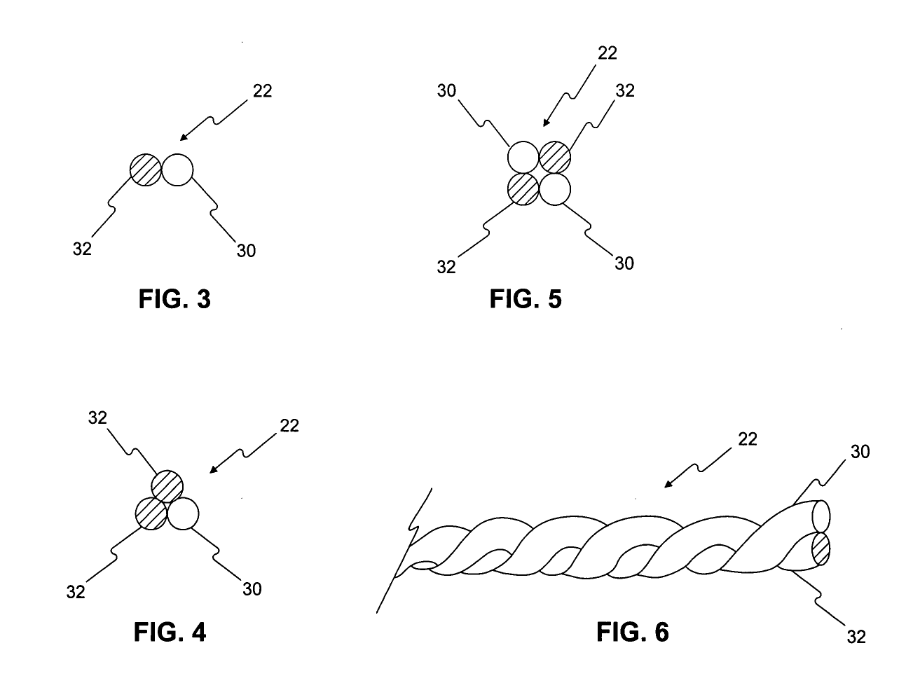

As shown in FIGS. 3-6, the legs 22 may be comprised of two or more discrete pieces of material, such as, but not limited to, discrete wires, fibers, strands, filaments, or like elongate structures. FIGS. 3-6 show, for example, wires 30 and fibers 32. The wires 30 may be made from any biocompatible metal or metal alloy, such as, for example, stainless steel, cobalt chromium, nickel, titanium, and nitinol. The fibers 32, on the other hand, may be made from any material having a relatively high resistance to laser energy. Such materials may include, for example, polymers and ceramics. The wires 30 and fibers 32 may be braided, intertwined, bonded, adhered, fused, or otherwise connected together to form a single leg 22, and each leg 22 may comprise at least one wire 30 and at least one fiber 32.

In an embodiment in which the legs 22 are braided, each leg 22 may be characterized by a desired number of twists per inch. In an exemplary embodiment, the wires 30 and fibers 32 may have at least ten twists per inch, and in an additional exemplary embodiment, the wires 30 and fibers 32 may have at least twenty twists per inch. It may be difficult to adhere, bond, fuse, or otherwise connect metal wires 30 to the ceramic and/or polymeric fibers 32 discussed above. Accordingly, braiding or intertwining the wires 30 and fibers 32 together may make it easier to manufacture the exemplary baskets 4 of such an embodiment. In addition, a braided structure provides a strong mechanical connection between wires 30 and fibers 32 without the use of an additional adhesive material or layer between the wires 30 and fibers 32.

FIG. 6 further illustrates the exemplary braided leg 22 of FIG. 2 having one metal wire 30 and one ceramic fiber 32. Each leg 22 may comprise any combination of wires 30 and fibers 32 useful in forming a basket 4 having desired strength, flexibility, shape memory, laser resistance, and/or other characteristics. The wires 30 of each leg 22 may impart, for example, strength and/or shape memory to the basket 4. Such characteristics may assist in retrieving a stone or other calculus from within a patient. At least the strength and/or flexibility of each leg 22 may depend on the type of metal used as well as the length, cross-sectional shape, diameter, and/or gauge of the wire 30. The fibers 32 of each leg may be capable of withstanding much higher temperatures than the metal wires 30 and may be resistant to laser energy used in conventional laser lithotripsy devices. Thus, the fibers 32 may assist in maintaining the integrity of the basket 4 if the metal wires 30 were accidentally damaged during a lithotripsy procedure.

In an exemplary embodiment, each leg 22 may include at least two ceramic fibers 32 and a single metal wire 30 (FIG. 4). In another exemplary embodiment, each leg may include a pair of ceramic fibers 32 and a pair of metal wires 30 (FIG. 5). The number of fibers 32 and wires 30 used in each leg 22 may affect the strength, flexibility, laser resistance, and other basket characteristics discussed above. For example, a leg 22 including a relatively large number of metal wires 30 may be stronger than a leg 22 having fewer wires 30. Moreover, a leg 22 including a relatively large number of fibers 32 may be more resistant to laser energy than a leg 22 having fewer fibers 32.

In an exemplary embodiment of the present disclosure, the diameter of the ceramic fibers 32 may be substantially the same as the diameter of the metal wires 30. In another exemplary embodiment, the diameter of the wires 30 may be different than the diameter of the fibers 32. Ceramic fibers 32 having a relatively large diameter may be braided with metal wires 30 having a relatively small diameter to impart desirable heat resistance characteristics to the resulting basket 4. Alternatively, metal wires 30 having a relatively large diameter may be braided with ceramic fibers 32 having a relatively small diameter to impart desirable strength characteristics to the basket 4.

The positioning of the wires 30 relative to the fibers 32 in each respective leg 22 may impart beneficial characteristics to the basket 4. For example, in an embodiment in which the legs 22 are not braided, it may be desirable to position fibers 32 on an inner surface of the leg 22 (or other surfaces that may be exposed to laser energy during lithotripsy) to increase the laser-resistant properties of the leg 22. The wires 30 and fibers 32 may include any cross-sectional shape known in the art, such as, for example, cylindrical, flat, square, semicircular, arced, D-shaped, rectangular, or ovular.

At least the metal wire 30 component of each leg 22 may be cold worked or heat processed to form a shape in memory. The shape may be fully formed when the basket 4 is fully expanded. Alternatively, the shape may be partially formed when the basket 4 is partially expanded. The resulting basket 4 may be any shape useful in capturing and/or retrieving a stone. Such shapes may include, for example, substantially spherical and substantially lemon-shaped. Although not shown, at least a portion of at least one of the legs 22 of the basket 4 may be coated with, for example, a layer of PTFE, ePTFE, polyvinylethylene, or other material to further protect the legs 22 during processes such as, for example, laser lithotripsy.

The elongate member 6 may be connected to the legs 22 in any conventional way. In an exemplary embodiment, the elongate member 6 may be formed from the wires 30 and fibers 32 of each leg 22. In such an embodiment, the elongate member 6 may be formed of, for example, braided legs 22, and the elongate member 6 may itself be braided. Alternatively, in another exemplary embodiment in which the legs 22 are braided, the elongate member 6 may be comprised of the unbraided wires 30 and/or fibers 32 of each leg 22.

Referring again to FIG. 1, the legs 22 may be joined at the distal end 26 of the basket 4 to form the atraumatic tip 24. The atraumatic tip 24 may be blunt, rounded, flat, smooth, knotted, looped, or any other atraumatic shape known in the art. The atraumatic tip 24 may be sized to assist in the capture and retrieval of stones within the body. For example, the atraumatic tip 24 may be as small as possible so as not to interfere with the capture and retrieval of the targeted stone. The atraumatic tip 24 may be composed of the same material as the legs 22, and the atraumatic tip 24 may be formed by soldering, welding, cementing, knotting, tying, looping, braiding, and/or otherwise connecting the legs 22 together. In an exemplary embodiment, the atraumatic tip 24 may also comprise a cap.

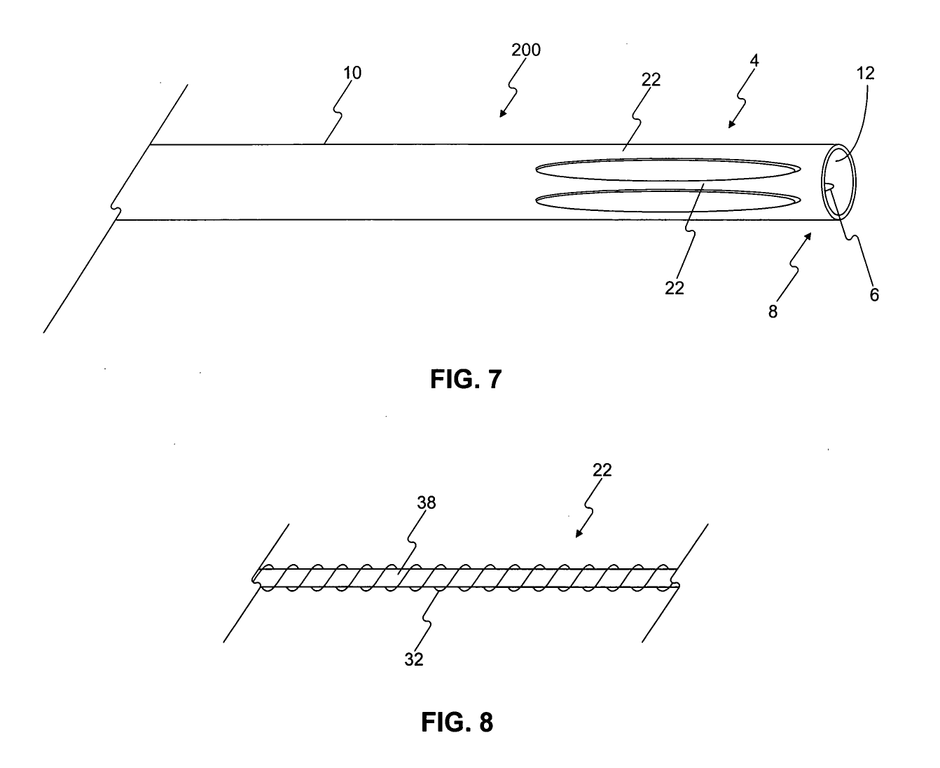

As shown in FIG. 7, in an additional exemplary embodiment of the present disclosure, a retrieval device 200 may include basket legs 22 formed by laser cutting, etching, stamping, and/or otherwise removing material from a sheath 10. As in the embodiments described above, the sheath 10 may be formed from, for example, a rod, tube, hypotube, cannula, stent, or other piece of biocompatible material or combination of biocompatible materials known in the art. Such materials may include, but are not limited to, polyamide, PEBAX, stainless steel (such as 300 and 400 series), cobalt chromium, nickel, titanium, nitinol, thermoforming plastic, polytetrafluoroethylene (“PTFE”), and expanded polytetrafluoroethylene (“ePTFE”). In the exemplary embodiment shown in FIGS. 7 and 8, the sheath 10 may comprise at least one of the above metals or alloys.

The device 200 may also include an elongate member 6 connected to a distal end 8 of the sheath 10. The elongate member 6 may be connected to the sheath 10 in any conventional way and may be formed from any of the materials described above with respect to the elongate member 6 of other exemplary embodiments. The elongate member 6 may be movably disposed within a lumen 12 of the sheath 10 and may be configured to expand the basket 4 when moved in the proximal direction relative to the sheath 10. As the elongate member 6 is moved in the proximal direction, the legs 22 may bow or otherwise deflect radially outward to form an expanded position of the basket 4. When the elongate member 6 is extended in the distal direction, the legs 22 may return to the position shown in FIG. 7, thereby forming a collapsed basket position.

In an additional exemplary embodiment, a sheath 10 (not shown) may be provided to assist in expanding and collapsing the basket 4 of the device 200. For example, moving the sheath 10 over the basket 4 may collapse the basket 4 within the sheath 10. The basket 4 may be configured to open when the sheath 10 is retracted. The legs 22 may be formed of materials having shape memory characteristics useful in forming both the expanded and the collapsed basket positions.

The legs 22 of the device 200 may be comprised of two or more discrete pieces of material. As shown in FIG. 8, the legs 22 may comprise strips 38 of the sheath 10, and fibers 32. The fibers 32 may be made from any material having a relatively high resistance to laser energy. As described above, such materials may include, for example, polymers and ceramics. The strips 38 and fibers 32 may be braided, intertwined, bonded, adhered, fused, or otherwise connected together to form a single leg 22, and each leg 22 may comprise at least one strip 38 and at least one fiber 32.

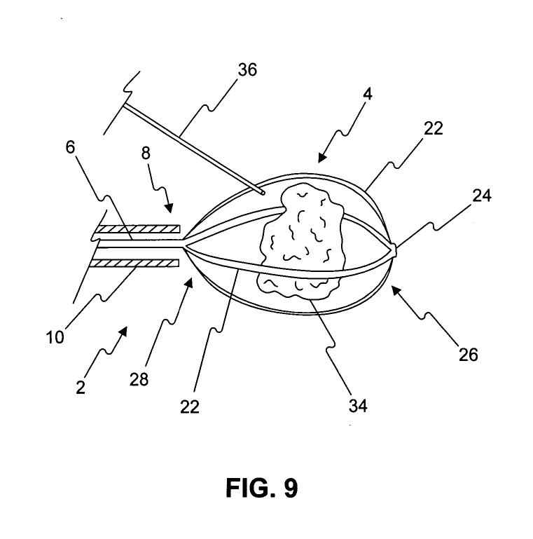

As illustrated in FIG. 9, at least some aspects of the present disclosure may be used, for example, to retrieve a stone 34, calculus, or other material from any location within the body, such as, for example, in the urinary tract of the patient. The device 2 may be inserted through the urethra of the patient or, alternatively, the device 2 may be inserted percutaneously to a treatment site. The treatment site within the body may correspond to the location of a targeted stone.

The stone 34 targeted for retrieval may be a kidney stone, a struvite, a uric acid stone, a cystine stone, or other solid deposit commonly removed from a body structure or passageway within the body. Such stones 34 may contain various combinations of chemicals, including, but not limited to, calcium, oxalate, and phosphate. The stone 34 may be of any size and could have a length or diameter of approximately 1 mm to 12 mm. These lengths and diameters are merely exemplary, and aspects of the present disclosure may assist in the retrieval of stones larger or smaller than those discussed herein. Stones 34 may be of any shape and could be, for example, flat, round, smooth, or jagged. The device 2 may retrieve stones 34 that are both impacted and free floating.

The device 2 may be advanced to the treatment site through an access sheath, stent, or other access or dilatation device known in the art. In addition, the device 2 may be used in conjunction with an endoscope (not shown) or other type of intracorporeal scope known in the art. The endoscope may advance through the body over a guidewire to the treatment site. Alternatively, the endoscope may be independently fed to the treatment site without the use of a guidewire. Once the treatment site has been reached, the device 2 may be fed through an access port of the endoscope to gain access to the stone 34.

While being advanced to the treatment site, the basket 4 of the device 2 may be at least partially, and preferably fully, enclosed within the elongate member 6. This configuration (shown in FIG. 2) may minimize the size of the device 2 and may assist in advancing the device 2 through the endoscope. Upon exiting the endoscope and accessing the stone 34, the user may extend at least a portion of the basket 4 from the distal end 8 of the sheath 10. The basket 4 may then be manipulated relative to the stone 34 so as to capture the stone 34 within the basket 4. Once captured, the stone 34 may be retrieved by removing the device 2 from the body of the patient.

If, however, the targeted stone 34 is larger than approximately 3 mm, the stone 34 may be too large to be safely removed from the body. In these situations, the user may use the device 2 to capture and assist in immobilizing the stone 34. The user may then perform a stone-reduction process, such as, for example, laser lithotripsy, to break up or otherwise reduce the size of the stone 34. The device 4 may act as a backstop during such a process and may assist in preventing particles of the stone 34 from migrating or escaping from the treatment site during or after the stone-reduction process.

As shown in FIG. 9, a laser fiber 36 or other conventional device may be used to break up the stone 34. Such a device may be difficult to use accurately within difficult-to-reach spaces, such as, for example, body structures or passageways within the body of a patient. Thus, it may be common to accidentally direct laser and/or other energy onto the legs 22 of the basket 4 while performing the stone-reduction process. The leg configurations discussed above, such as, for example, braided legs 22 including metal wires 30 and ceramic fibers 32, may assist in maintaining the integrity of the basket 4 if one or more of the legs 22 were damaged. In particular, the ceramic fibers 32 in each leg 22 may assist in holding a damaged leg 22 together in one piece if a metal wire 30 of the leg 22 was severed.

The laser fiber 36 may be fed through an access port of an endoscope and may be activated and controlled by the user to reduce the size of the stone 34 or to fragment it into smaller pieces. A proximal end of the laser fiber 36 may connect to a power source (not shown). Once the stone 34 has been reduced, the device 2 may act as a sweeping device to sweep stones and stone particles obtained from the reduction process out of the body.

Other embodiments of the invention will be apparent to those skilled in the art from consideration of the specification and practice of the invention disclosed herein. For example, foam, mesh, webbing, or some other material may be attached to a number of the legs 22 to assist in the capture, retrieval, or immobilization of a stone 34. It is intended that the specification and examples be considered as exemplary only, with the true scope and spirit of the invention being indicated by the following claims.

What is claimed is:

1. A medical device, comprising:
a sheath defining a lumen;
an elongate member disposed within the lumen; and
a basket connected to a distal end of the elongate member and being retractable within and extendable from the lumen, the basket including a plurality of legs, each of the plurality of legs comprising a first elongate structure of a first material braided with a second elongate structure of a second material, the second material having a resistance to laser energy greater than a resistance to laser energy of the first material.

2. The medical device of claim 1, wherein the first material comprises a metal.

3. The medical device of claim 2, wherein the metal is one of nitinol, stainless steel, cobalt chromium, nickel, and titanium.

4. The medical device of claim 3, wherein the first elongate structure is a wire.

5. The medical device of claim 1, wherein the second material is not a metal.

6. The medical device of claim 5, wherein the second material comprises one of a ceramic material and a polymer.

7. The medical device of claim 6, wherein the second elongate structure is a fiber.

8. The medical device of claim 1, wherein the basket forms a predetermined shape in an expanded position.

9. The medical device of claim 8, wherein the predetermined shape is one of substantially spherical and substantially lemon-shaped.

10. The medical device of claim 1, further including an atraumatic tip formed at a distal end of the basket.

11. The medical device of claim 1, wherein the elongate member comprises the first elongate structure and the second elongate structure of at least one of the plurality of legs.

12. The medical device of claim 1, wherein the basket comprises at least three legs.

13. The medical device of claim 1, wherein at least one of the plurality of legs comprises at least two ceramic fibers and at least one metallic wire.

14. The medical device of claim 1, wherein at least one of the plurality of legs comprises at least two ceramic fibers and at least two metallic wires.

15. A method of removing matter from the body of a patient, comprising:
providing a medical device including a sheath defining a lumen, an elongate member disposed within the lumen, and a basket connected to a distal end of the elongate member and being retractable within and extendable from the lumen, the basket including a plurality of legs, each of the plurality of legs comprising a first elongate structure of a first material braided with a second elongate structure of a second material, the second material having a resistance to laser energy greater than a resistance to laser energy of the first material;
advancing the medical device to a treatment site within the body of the patient;
capturing the matter within the basket of the device;
reducing the size of the matter; and
removing the medical device from the body of the patient.

16. The method of claim 15, further including immobilizing the matter with the plurality of legs of the basket.

17. The method of claim 15, further including advancing a laser fiber to the treatment site to assist in reducing the size of the matter.

18. The method of claim 15, further including preventing particles of the matter from escaping from the treatment site with the plurality of legs of the basket.

19. The method of claim 15, further including sweeping at least a portion of the matter from the treatment site with the plurality of legs of the basket.

20. The method of claim 15, wherein the matter is one of a kidney stone, a struvite, a uric acid stone, a cystine stone, and a solid deposit.

21. The method of claim 15, wherein the first material comprises a metal.

22. The method of claim 21, wherein the metal is one of nitinol, stainless steel, cobalt chromium, nickel, and titanium.

23. The method of claim 22, wherein the first elongate structure is a wire.

24. The method of claim 15, wherein the second material is not a metal.

25. The method of claim 24, wherein the second material comprises one of a ceramic material and a polymer.

26. The method of claim 25, wherein the second elongate structure is a fiber.

27. A medical retrieval device basket, comprising:
a plurality of interconnected legs, each of the legs including a first material braided with a second material, the second material having a resistance to laser energy greater than a resistance to laser energy of the first material.

28. The medical retrieval device basket of claim 27, wherein the first material comprises a metal.

29. The medical retrieval device basket of claim 28, wherein the metal is one of nitinol, stainless steel, cobalt chromium, nickel, and titanium.

30. The medical retrieval device basket of claim 27, wherein the second material is not a metal.

31. The medical retrieval device basket of claim 30, wherein the second material comprises one of a ceramic material and a polymer.

32. The medical retrieval device basket of claim 27, wherein the basket forms a predetermined shape in an expanded position.

33. The medical retrieval device basket of claim 32, wherein the predetermined shape is one of substantially spherical and substantially lemon-shaped.

34. The medical retrieval device basket of claim 27, further including an atraumatic tip formed at a distal end of the basket.

35. The medical retrieval device basket of claim 27, wherein the basket comprises at least three legs.

36. The medical retrieval device basket of claim 27, wherein each of the plurality of legs comprises at least two strands of the second material braided with at least one strand of the first material.

37. The medical retrieval device basket of claim 27, wherein each of the plurality of legs comprises at least two strands of the second material braided with at least two strands of the first material.

38. The medical retrieval device basket of claim 27, wherein the first material of each of the plurality of legs comprises a strip formed from a sheath.

39. The medical retrieval device basket of claim 38, wherein the strip is formed by removing at least a portion of the sheath.

40. The medical retrieval device basket of claim 38, wherein the second material comprises one of a ceramic material and a polymer.

41. The medical retrieval device basket of claim 27, wherein the first material is a metal wire and the second material is a non-metal strand.
